SEQUENCE LISTING (12 sequences)

SEQ ID NO: 1 - DNA, 24 bp, Artificial Sequence
  Primer for TC22
ctgagtttgctgagagatcggtag

SEQ ID NO: 2 - DNA, 23 bp, Artificial Sequence
  Primer for TC 22
aggtcagtggtgtgagcagtaag

SEQ ID NO: 3 - DNA, 24 bp, Artificial Sequence
  Primer for hTM5
gataaactcaaggaggcagagacc

SEQ ID NO: 4 - DNA, 22 bp, Artificial Sequence
  Primer for hTM5
gactgggcgttctacatctcat

SEQ ID NO: 5 - DNA, 22 bp, Artificial Sequence
  Primer for Cox-2
ctcaggcagagatgatctaccc

SEQ ID NO: 6 - DNA, 22 bp, Artificial Sequence
  Primer for Cox-2
gtctggaacaactgctcatcac

SEQ ID NO: 7 - DNA, 24 bp, Artificial Sequence
  Primer for P53
gggagtaggacataccagcttaga

SEQ ID NO: 8 - DNA, 24 bp, Artificial Sequence
  Primer for P53
cttccctggttagtacggtgaagt

SEQ ID NO: 9 - DNA, 20 bp, Artificial Sequence
  Primer for CDK2
ggccatcaagctagcagact

SEQ ID NO: 10 - DNA, 20 bp, Artificial Sequence
  Primer for E2F
ccatctcagcaaagatgcag

SEQ ID NO: 11 - DNA, 20 bp, Artificial Sequence
  Primer for E2f
atgttttcctgtgccctgag

SEQ ID NO: 12 - DNA, 20 bp, Artificial Sequence
  Primer for E2F
atctgtggtgagggatgagg

(19) United States
(12) Patent Application Publication
(10) Pub. No.: US 2010/0304422 A1
(43) Pub. Date: Dec. 2, 2010

(54) In-Vitro Model of the Transformation of Metaplasia to Neoplasia

(76) Inventor: Kiron Das, Basking Ridge, NJ (US)

Correspondence Address: University of Medicine and Dentistry of New Jersey;Office of Patents and Licensing, Suite 2100, One worlds Fair Drive, Somerset, NJ, 08875, US

(21) Appl. No.: 12/731,995

(22) Filed: Mar. 25, 2010

Related U.S. Application Data
(60) Provisional application No. 61/211,301, filed on Mar. 26, 2009.

(51) Int. Cl. C12Q 1/02

(52) U.S. Cl. 435/29

(57) ABSTRACT

This invention relates to a novel in-vitro model of transformation of a benign Barrett's cell line to a neoplastic cell line following repeated exposure to acid and bile over the course of about 65 wks, and to the process of using the model to study the transformation.

### CROSS-REFERENCE TO RELATED APPLICATION

The instant application claims 35 U.S.C. §119(e) priority to U.S. Provisional Patent Application Ser. No. 61/211,301 filed Mar. 26, 2009, the disclosure of which is incorporated herein by reference.

### STATEMENT REGARDING FEDERALLY SPONSORED RESEARCH OR DEVELOPMENT

This invention was produced in part using funds obtained through grant RO1DK063618 from the NIDDK/NIH. The federal government may have certain rights in this invention.

This application relates to an in-vitro model of the transformation of metaplasia to neoplasia and to the use of the model in the study of the transformation of metaplasia to neoplasia.

Barrett's epithelium (BE) is a columnar metaplasia at the squamocolumnar junction of the distal esophagus secondary to chronic gastroesophageal reflux disease (GERD). Epidemiological studies indicate a strong relationship between GERD and esophageal adenocarcinoma (EAC). The incidence of EAC has the highest rate of rise amongst all cancer and increased almost 6-fold over the past few decades in the United States and in Western Europe. BE is a major risk factor for malignant transformation being 30- to 125-fold higher in GERD patients complicated with BE. Cancers develop in the metaplastic epithelium through a series of changes associated with exposure to acid and bile promoting DNA damage-mediated genetic alterations. In BE, such morphological changes can be recognized by pathologists as metaplasia→dysplasia→carcinoma. With repeated damage to critical, growth-regulating genes in the dysplastic cells, malignant clones emerge. The utility of extensively mutated malignantly transformed cells or cancer cells as models for understanding the development of Barrett's esophagus and the metaplasia→dysplasia→carcinoma sequence is limited as they have already acquired mutations and other changes that may not accurately reflect the process in vivo. Thus there remains a need for an improved model for study.

A novel in-vitro model of transformation of a benign Barrett's cell line to a cell line that formed tumors in nude mice has now been developed. The transformation followed repeated exposure to acid and bile over the course of 65 wks.

### BRIEF DESCRIPTION OF THE DRAWINGS

FIG. 1 shows morphological changes in BAR-T cells following acid and bile treatment at different times.

FIG. 2 A shows foci formation by acid and bile induced transformed foci in BAR-T cells and no foci formation in untreated cells.

FIG. 2B shows growth analysis of A+B treated BAR-T cells untreated cells at 65+ weeks

FIG. 3 shows colony formation in soft agar by A+B treated BAR-T cells at 65 weeks (B) & 82 weeks (C and D). (E) shows progressive, quantitative increase of colony formation (both small and large over time) in soft agar with A+B treated BAR-T cells.

FIG. 4 (A) shows TC22 mRNA expression in A+B treated BAR-T cells increased by 3-fold as compared to untreated cells at 22 weeks onward up to 62 weeks. (B) shows expression of similar genes as shown in panel A remained mostly unchanged in BAR-T cells exposed to Acid (pH4) alone up to 34 weeks.

FIG. 5 (A) shows P53 protein expression was measured in A+B treated (BAR/A+B) and untreated BAR-T cells (BAR/C) at 18, 48, and 64 weeks (W) by Western blot analysis. (B) P53 protein expression estimated by quantitative western blot assay in A+B treated BAR-T cells normalized against untreated control cells grown in parallel at different time points. (C) TC22 protein expression in A+B treated and untreated BAR-T cells at 64 wks by indirect Immunofluorescence Assay using anti-TC22 monoclonal antibody (TC22-4, IgG isotype).

We hypothesized that prolonged exposure of these benign cells to acid and bile (A+B) may cause transformation and extended the exposure period up to one year and beyond. We investigated if prolonged exposure of BAR-T cells to A+B confers a tumorigenic phenotype as determined by morphological changes, molecular changes, anchorage-independent growth and formation of tumors in nude mice.

Indeed, in this systematic, prospective analysis over the course of 65 weeks, we have demonstrated that following daily exposure to A+B for a brief period of 5 min/day, BAR-T cells showed progressive morphological, molecular and biological changes. These changes are consistent with a transformed phenotype. Anchorage independent behavior was observed as foci formation and growth on soft agar in the A+B treated cells. The number of discrete colony formation in soft agar was progressively increased with longer exposure to A+B only after 58 weeks.

It is believed that cancer arises in BE through a multi-step sequence of events initiated, most likely, by chronic GERD that leads to metaplasia→dysplasia →adenocarcinoma. A large nationwide population-based case-controlled study from Sweden found a strong correlation of adenocarcinoma of the esophagus with the chronicity of GERD. Since BE was associated with esophageal adenocarcinoma many studies focused on the understanding of the molecular aberrations characteristic to this progression. Early studies described the presence of p53 mutations in esophageal adenocarcinoma and increased frequency of p53 mutations during the progression of metaplasia→dysplasia→carcinoma sequence. Furthermore, there is increased frequency of non-random chromosomal aberrations associated with progression of disease, including loss of heterozygosity in several key regions, e.g. 17p, 9p. The frequency of 17p LOH increased with degree of loss of p16. Combined p53 mutation and LOH of 17p (the p53 locus) results in loss of p53 tumor suppressor function, propensity to develop genomic instability, emergence of aneuploid subclones, and disabled G1 checkpoint leading to clonal expansion of abnormal cells. Dysregulated cell cycling through changes in p16 and cyclin D1 expression associated with increased proliferative potential, a process normally offset by intact p53-mediated apoptotic mechanisms in esophagitis, are unbalanced to inhibition of apoptosis in high grade dysplasias and adenocarcinomas. Evidence supports the association between 17p LOH and p53 mutations with increased risk of dysplasia and/or esophageal adenocarcinoma.

The in vitro system described herein is unique in that BAR-T cells are diploid, exhibit non-neoplastic properties, with intact p53 and p21 cell cycle checkpoints but have lost p16 expression. BAR-T cells treated with A+B for 22 weeks demonstrate more than 2-fold increase in p53 gene expression which begins to decline after 42 weeks and at 60+ weeks, there is no change in p53 gene expression between A+B treated and untreated cells. However, western blot analysis shows that although p53 levels increase with treatment relative to control cells at week 42, this increase is smaller than at earlier weeks. The p53 protein level in control cells varied slightly between measurements, but the increase in p53 in A+B treated BAR-T cells relative to the control cells lessened with longer exposure times until there was no detectable change between the two lines at 64 weeks. The relative lag in p53 protein level in comparison to gene expression may reflect stabilization of protein that occurs with cell stress in the absence of altered gene expression. This is known to occur with activation of several upstream kinases. Despite the lag at 42 weeks, at subsequent time points BAR-T cells treated with A+B show both a decrease in protein levels and a lack of increased gene expression. Loss of A+B induced p53 expression was noted at time points associated with morphologic changes. This may be explained by either acquired mutation in p53 and/or loss of heterozygosity at 17p that either leads to reduced stabilization of p53 or increased degradation. Independent of the mechanism by which p53 is lost reduced levels of p53 translate into loss of activation of the downstream effectors and regulators p21, PERP, and MDM2. Loss of PERP activation, suggests a diminished capacity to induce apoptosis, and p21 is necessary to induce cell cycle arrest. Therefore prolonged BAR-T exposure to A+B, loss of inducible p53 and target gene expression suggest dysregulation within the p53 pathway and these changes paralleled with contact inhibition cell behavior as witnessed in soft agar assays are consistent with transformation. Acid exposure has been reported to have anti-proliferative effect in non neoplastic Barrett's cells (BAR-T) although it has pro-proliferative effect on Barrett's associated adenocarcinoma cells, The increased proliferation witnessed in A+B treated cells after 65 weeks as compared to the untreated counterparts coupled with an increase in expression of E2f and CDK2 expression and suppressed p21 are suggestive of the neoplastic Barrett's phenotype. Cells with such molecular changes may result in accumulation of genetic changes and increased genomic instability. We hypothesize that dysregulation of the p53 pathway contributes to the transformed phenotype. This in-vitro model allows identification of dynamic molecular changes in various pathways occurring longitudinally.

TC22 is a neoplastic marker expressed by transformed cells but not by normal epithelial cells. In colonic neoplasia, a progressive increase of TC22 expression was observed in benign adenomas (35%) to mild dysplasia (57%) and severe dysplasia (100%). In a pilot study, we observed that the frequency of mAb TC22 expression in the gastric intestinal metaplasia associated with gastric cancer was 86% (19 of 22). In this cell culture model, there was a significant increase (300%) in the expression of TC22 when compared to the untreated cells. However, normal epithelial tropomyosin isoform 5 (hTM5) was unchanged compared to the untreated cells.

Although BAR-T cells have lost p16, untreated cells do not display the malignant phenotype. One might conclude that loss of p16 itself is not sufficient to recapitulate transformation. The introduction of hTERT for immortalization is unlikely to transform these cells alone but is sufficient to propagate cells long term. In the hTERT immortalized breast+epithelial BPE+HPE cells described by Ince et al (48) only upon introduction of K-RAS and large antigens, do these epithelial cells change morphology and take on a transformed phenotype. It is likely that additional molecular alterations such as dysregulation of the p53 pathway have led to the phenotypic change particularly growth in soft agar, a hallmark of transformation observed in BAR-T treated cells after prolonged A+B treatment. Additional studies utilizing this novel in-vitro model system will provide further understanding of the molecular events that lead to metaplasia→dysplasia→carcinoma following acid and bile exposure.

Acid and bile, the two primary components of gastroesophageal refluxate, act synergistically in inducing mucosal injury. Molecular events resulting from GERD in humans have been studied in esophageal biopsies and adenocarcinoma cell lines. A single pulse of either bile or acid independently increases cell survival and proliferation, as well as decreases apoptosis, in vitro, via activation of the mitogenactivated protein kinase pathways and down regulation of the caspase cascade and ERK pathway. Similarly, bile acids have been shown to stimulate proliferation of BE in an acid-dependent fashion. Both acid and bile have been proposed to promote intestinal-type differentiation in esophageal keratinocytes by inducing the transcription factors NF-kB and Cdx-2. Acid has been shown to induce villin expression in normal esophageal biopsy tissues grown in organ cultureand bile, at neutral pH, to cause DNA damage in esophageal cell lines. Both acid and bile can cause oxidative intracellular production of reactive oxygen species in esophageal cells and the anti-oxidants can inhibit acid- and bile-induced DNA damage. In most of these experiments, acid and/or bile exposure have been only short-term for a few minutes or hours. There are, however, no reports of phenotypic and molecular changes resulting from long-term (weeks to months) episodic exposure to acid and bile on keratinocyte or benign BE cells.

We developed a monoclonal antibody (mAb) 7E12H12, also known as mAb Das-1, that specifically reacts with human colon epithelial cells (both goblet and non-goblet cells), but not with any other parts of the gastrointestinal tract, including small intestine, gastric and esophageal mucosa. The antibody, however, reacts with specialized columnar epithelium/Barrett's epithelium (SCE/BE) with almost 100% sensitivity and specificity. These observations have been validated by three independent groups of pathologists/gastroenterologists. Subsequently, we reported that in about 15-30% of patients with gastroesophageal reflux disorders (GERD), mAb Das-1 reacts with discrete non-goblet columnar cells at the esophago-gastric junction (EGJ) in the absence of histological BE or focal goblet cell (GC) metaplasia (IM), suggesting the presence of a “Pre-Barrett's metaplasia”. We further observed that non-GC “cardia type epithelium” (CE) associated with BE also frequently (65%) react with mAb Das-1).

From a cDNA library prepared from a colon cancer cell line, T84, we have isolated and sequenced a novel tropomyosin (TM) isoform, TC22 that is expressed by transformed epithelial cells and colon tumor tissue but not by normal epithelial cells. Normal epithelial cells express hTM isoform 5 (hTM5). TC22 is identical to hTM5 apart from the C-terminal domain amino acids 222-247 coding the exon 9. TC22 is an alternatively spliced product of hTM5. Almost 100% of colon cancer tissue showed TC22 expression where as normal colon tissue and hyperplastic polyps did not (p<0.0001). TC22 expression progressively increased in benign adenomatous polyps (35%) and polyps with mild and severe dysplasia (57% and 100% respectively). Jaiswal et al developed a telomerase-immortalized, non-neoplastic, human BE cell line (BAR-T). BAR-T cells were received from University of Texas Southwestern medical Center at Dallas (kindly provided by Rhonda F Souza, M. D.). The BAR-T cells have been sustained in culture beyond 200 populations doubling, while maintaining diploid chromosome number and exhibiting non-neoplastic properties such as contact inhibition and anchorage-dependent growth. They also maintain various histological markers and appropriate expression of p21 and p53. BAR-T cells react with mAb Das-1 antibody indicating colonic phenotype, repeated acid and bile exposure up to 6 weeks appears to induce expression of colonic phenotype (mAb Das-1 positive cells) in this cell line (34). The BAR-T cell line comprised of 35±5.2% CK8/18, 32±3.5% mAb Das-1, 9.5±3% CK4 and 4±2.5% p75NTR-positive cells. Single exposure to acid and or bile did not change cell phenotypes. However, chronic treatment for at least 2 weeks significantly enhanced (P<0.05) the expression of colonic phenotype and CK8/18-positive cells, as evidenced by FACS analysis. Bile salt at pH 4 and bile salt followed by acid (pH 4) in succession were the strongest stimulators (P<0.01) for induction of colonic phenotype cells. Squamous (CK4+) phenotype did not change by the treatments.

### Materials and Methods

Cell line medium and cell culture—BAR-T cells were grown in special supplemented keratinocyte medium (KBM2) from Cambrex Bioscience (East Rutherford, N.J., USA), as per the protocol described by Jaiswal et al. Hydrochloric acid (A) was used to adjust the pH of the culture medium to experimental conditions. The bile acid, glycochenodeoxycholic acid, GCDA (Sigma, St. Louis, Mich., USA), was diluted to optimum working concentration of 200 μM (B) with the culture medium adjusted to pH4 (A+B). 0.1×10₆ cells growing on six-well plates were incubated in A or A+B for 5 min in 24 h. For chronic exposure, cells were exposed for 5 min everyday to A only for up to 34 weeks and A+B for up to 84 weeks. No treatment was done on the day the cells were passed. The time was optimized from similar studies, showing that 5 min was sufficient for induction of signal transduction pathways regulating cellular machinery without cell damage. The cells were rinsed with phosphate buffered saline (PBS) before and after incubation with desired treatment medium. The control untreated cells were grown in parallel in the special medium as mentioned above at pH7.4. A portion of the cells were harvested every 4-6 weeks.
Morphological Changes and Growth analysis—BAR-T cells that were exposed to A+B at various weeks were plated at 1×10₅ cells per well in 12 well plates and counted in triplicate using a Beckman Coulter Counter (Vi-CELL1.00). Cells were counted periodically and morphological changes were examined weekly.
Foci assay: Cells were plated at 5×10₅ cells per 100 mm dishes (35). Untreated cells grown in parallel were also plated. Cells were grown for 4-5 weeks with regular medium changes beyond 100% confluency, fixed with 10% methanol, 10% acetic acid solution, and stained with 20% ethanol, 0.4% crystal violet for 5 min.
Growth in soft agar: Each well of the six well plates was layered with base agar layer containing 0.8% agar in grown medium. Cells were plated in 0.4% agar in growth medium at a density of 6000 cells per well. 500 μl of growth medium were added on top of the agar. Four weeks after plating, the colonies were stained with Cell transformation detection assay substrate (Chemicon). Large (>1 mm) and small colonies (<1 mm) were visually identified, photographed and counted separately.
Molecular Changes: RT-PCR analysis—Total RNA was extracted using the Qiagen RNeasy Mini Kit per manufacturer's instruction. The RNase-Free DNase set (Qiagen) was used during RNA purification, cDNA was generated using Advantage RT-for-PCR kit, DNA contamination was tested for by PCR of the RT samples. All runs were accompanied by a negative control which included all reagents, except cDNA. Expression of target mRNA was normalized with respect to β actin using the ΔΔCT method.

All samples underwent 40 cycles of denaturing at 95° C. for 15 seconds, annealing at 60° C. for 20 seconds, and extending at 72° C. for 20 seconds on the Roche Lightcycler using the Qiagen SYBR Green PCR kit. Gene expression assays that included primer and probe sets for Taqman assays for p21 (Hs00355782_m1), MDM2 (Hs00242813_m1) and PERP (Hs00751717_s1) genes were obtained from Applied Biosystems. Primers for other genes were as follows:

(1) TC22:
(SEQ ID NO: 1)
5′-CTG AGT TTG CTG AGA GAT CGG TAG-3′
and
(SEQ ID NO: 2)
5′-AGG TCA GTG GTG TGA GCA GTA AG-3′
(2) hTM5:
(SEQ ID NO: 3)
5′GAT AAA CTC AAG GAG GCA GAG ACC-3′
and
(SEQ ID NO: 4)
5′-GAC TGG GCG TTC TAC ATC TCA T-3′
(3) Cox-2:
(SEQ ID NO: 5)
5′-CTC AGG CAG AGA TGA TCT ACC C-3′
and
(SEQ ID NO: 6)
5′-GTC TGG AAC AAC TGC TCA TCA C-3′
(4) P53:
(SEQ ID NO: 7)
5′-GGG AGT AGG ACA TAC CAG CTT AGA-3′
and
(SEQ ID NO: 8)
5′-CTT CCC TGG TTA GTA CGG TGA AGT-3′
(5) CDK2:
(SEQ ID NO: 9)
5′-GGC CAT CAA GCT AGC AGA CT-3′
and
(SEQ ID NO: 10)
5′-CCA TCT CAG CAA AGA TGC AG-3′
(6) E2F:
(SEQ ID NO: 11)
5′-ATG TTT TCC TGT GCC CTG AG-3′
and
(SEQ ID NO: 12)
5′-ATC TGT GGT GAG GGA TGA GG-3′

Western blot analysis: The Western blot assay was performed according to published protocols. Thirty micrograms of total protein extracts from BAR-T cells were resolved on 4-20% gradient SDS-polyacrylamide gel (Invitrogen). The blots were then incubated with anti-p53 antibody (DO-1, Santacruz) and subsequently with HRPconjugated anti-mouse secondary antibody (Santacruz) and then developed by ECL reagent (Perkin-Elmer). The same blots were also incubated with an HRP-conjugated anti-GAPDH antibody (Santacruz) to determine protein loading.
Immunoperoxidase staining: Cytospin preparation of BAR-T cells was fixed and stained using the method previously described. Briefly, the cells were incubated with TC22-4 (IgG isotype) (cancer marker) overnight at 4° C. at 1:400 dilution followed by biotin-conjugated rabbit anti-mouse secondary IgM or IgG depending on the primary mAb (1:50) for 1 h, with avidin-biotin enzyme reagent for 30 min and finally with peroxidase substrate for color development.
Growth of cells as tumors in xenograft model: A+B treated BAR-T cells exposed for 65+ weeks were transplanted (10₇ cells per mouse) subcutaneously over the dorsum of athymic nu/nu mice. Untreated BAR-T cells grown in parallel for the same duration were also injected in a separate batch of nude mice. C85 colon cancer cells were injected in separate mice, as positive control.

### Results

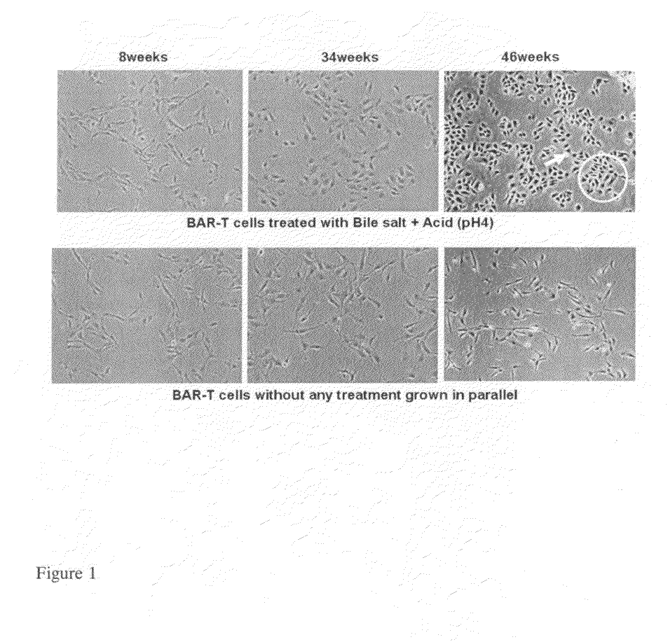

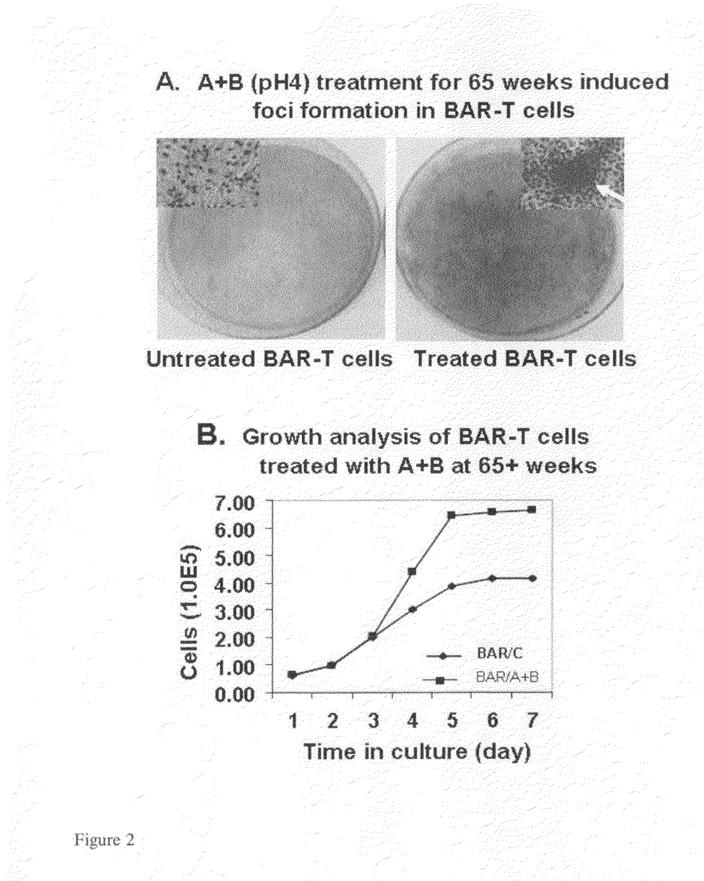

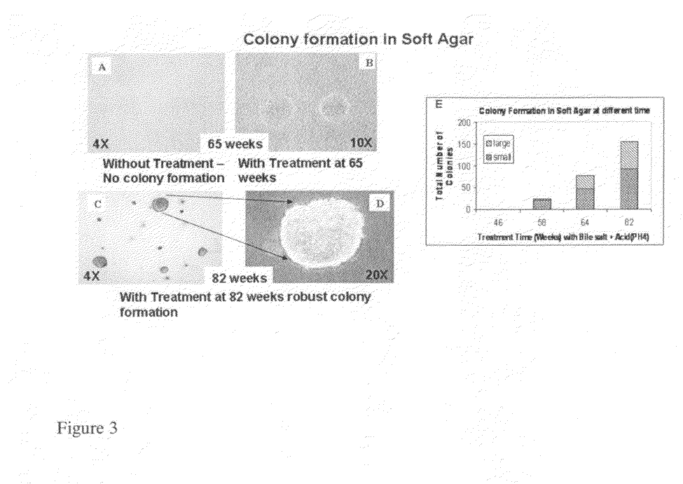

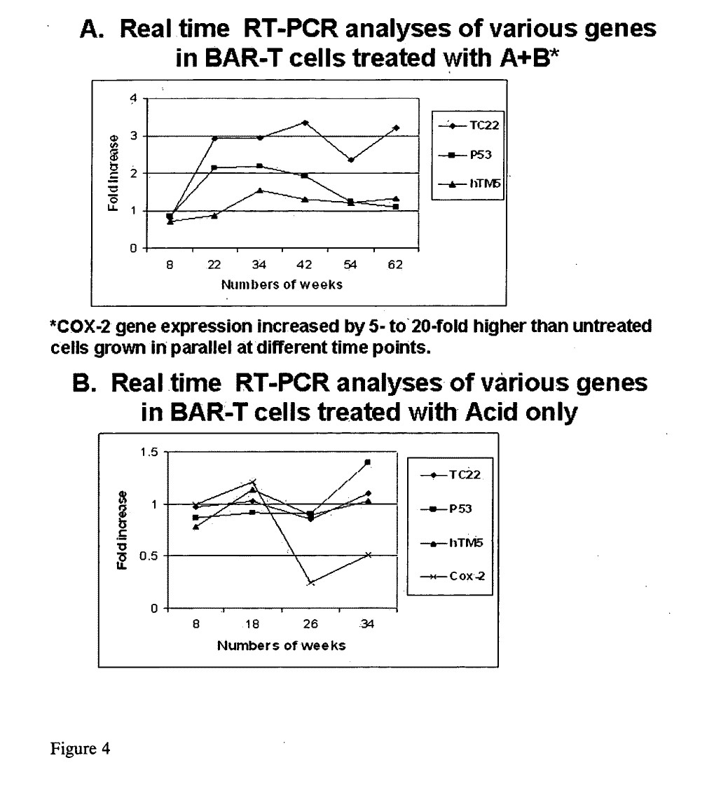

Morphological changes—Morphological changes between untreated and A+B treated cells were evident from 34 weeks onward, However, distinct phenotypic changes were observed at 46 wks (FIG. 1, upper panels) when A+B treated BAR-T cells grew as round or oval cells in clumps (circle) and displayed acini (arrow) like formation. Untreated cells remained spindle shaped and evenly dispersed on the culture plate (lower panels).
Foci assay—A+B treated BAR-T cells demonstrated loss of contact inhibition, a transformed phenotype with formation of foci in monolayer cultures at 65 weeks onward. A+B treated cells for 65 weeks gave rise to foci after 3 weeks of culture, whereas untreated BAR-T cells grown in parallel for the same length of time did not form any foci (FIG. 2A). Microscopic examination of the foci revealed multi-layered growth and crisscross morphology characteristics of transformed cells. Furthermore, the nuclei are also larger with a tendency to clump (FIG. 2A inset).
Growth analysis—The kinetics of cell growth was evaluated for A+B treated BAR-T cells and control cells at different time points. FIG. 2 shows BAR-T cells that had been exposed to A+B for more than 65 weeks. The same number of cells was plated on Day 0 for both groups. Growth was the same for both groups on days 1-3. However, A+B treated group demonstrated a 2-3 fold higher growth rate on day 4 onward as compared with control untreated cells grown in parallel (FIG. 2B).
Soft agar colony—Since initial morphological changes were seen at 46 weeks, soft agar colony assays were initiated and repeated subsequently every 4-8 weeks. No colony formation was observed at 46 weeks. At 58 weeks, we first observed colony formation in soft agar. Number of colonies both small (<1 mm) and large (>1 mm) progressively increased with longer exposure of BAR-T cells to A+B, up to 82 weeks (FIG. 3 B-D). FIG. 3 demonstrates the colonies, small and large, at 65 and 82 weeks. The percentage of both small and large; in particular large, colony formation in comparison to small colony formation increased with progressively longer duration of treatment with A+B (FIG. 3E). Colony formation was robust at 82 weeks (FIG. 3 C&D). BAR-T cells cultured in parallel without A+B treatment for the same length of time did not show any colony formation (FIG. 3A).
Molecular changes: Cox-2, TC22, hTM5, p53, CDK2 and E2F mRNA expression were analyzed by real time RT-PCR at 8 to 12 week intervals in cells exposed to A+B treated, as well as untreated cells grown in parallel (FIG. 4A). Cox-2 gene expression increased 10-fold at 22 weeks and varied between 5-20-fold higher than untreated cells up to 62 weeks (data not shown). TC22 gene expression increased at 22 weeks by 3-fold and maintained up to 62 weeks as compared to untreated cells (FIG. 4). However, hTM5 (normal epithelial tropomyosin isoform) gene expression did not change. P53 gene expression doubled at 22 weeks and maintained at this level up to 34 weeks. However, there was a sharp decline of p53 gene expression at 42 and 54 weeks and return to the baseline level (same as untreated cells) at 60+ weeks (FIG. 4). Changes in gene expression for p53 target genes p21, MDM2, and PERP in addition to the p53 gene were also measured by real-time RT-PCR. Repeated exposure of BAR-T cells to A+B resulted in a 2.2-fold induction of p53 at 34 weeks coinciding with the induction of the p53 target genes MDM2, PERP and p21, 6.4-, 4-, and 2.6-fold respectively at 34 weeks. Untreated cells did not show changes in expression of p53 or p53 related target genes. Similar measurements made at 58 and 65 weeks failed to show activation of p53 with continued treatment nor were there any measurable changes in expression of p53 target genes with the exception of very small changes in PERP at 58 and 65 weeks to 2.7- and 1.6-fold respectively. Expression of PERP declined in later weeks relative to that observed at 34 and 58 weeks. These data suggest that at 34 weeks, p53 was inducible with A+B exposure and its ability to activate genes involved in cell cycle arrest and apoptosis was still intact. However, more prolonged chronic exposure to A+B resulted in loss of p53 induction as well as p53 mediated regulation of target genes.

Chronic exposure to A alone did not show any morphological changes. As opposed to the treatment with A+B (FIG. 4A), BAR-T cells exposed to A alone up to 34 weeks did not show the above molecular changes particularly for the transformation marker, TC22 (31) although there was minor increase of P53 expression probably due to stress response (FIG. 4B). Further experiments with exposing BAR-T cells to A alone was therefore discontinued. Chronic exposure to B alone was not performed because in our initial study there was no appreciable effect of B alone on BAR-T cells up to 6 weeks.

Coincidentally, with increased proliferation (FIG. 2B) the expression of CDK2 and E2F mRNA also increased by 2.8 and 4.7 fold respectively in the A+B treated cells after 65 weeks when compared with the untreated controls.

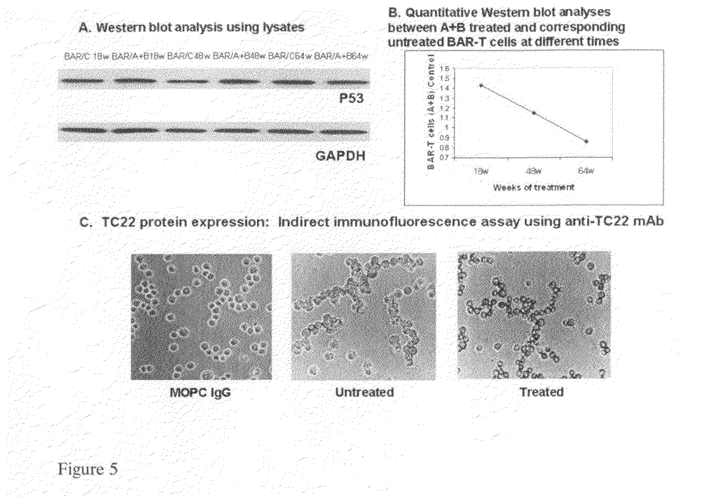

Protein expression—Western blot analysis of cell lysates from A+B treated BAR-T cells were compared to the control (untreated) BAR-T cells at different times of exposure (FIG. 5A). Image J software was used to compare the band intensity for both p53 and
GAPDH by western blot assay. There was no distinct pattern change of p53 expression in control cells. In general, at 18 weeks and 48 weeks, p53 expression in A+B treated BAR-T cells was higher than in that of control cells. Although 64 week control cells had slightly increased p53 relative to 18 weeks, there was no change in expression in treated cells as compared with control cells. Using band intensity values obtained from Image J, p53 expression in A+B treated BAR-T cells is normalized against control cells at the respective time points (FIG. 5B). P53 protein expression increased considerably at the initial stage of A+B exposure. However, at and after 48 weeks, p53 expression sharply declined to the baseline level.

TC22 protein expression increased as assessed by FACS analysis and by immunocytochemical stain with A+B treatment compared to untreated cells at 65+ weeks. The staining was cytoplasmic (FIG. 5C).

Tumor formation in nude mice—There was formation of tumor in three of three mice after 3 weeks of injection of 10₇ A+B treated (for 65+ weeks) BAR-T cells. Untreated BAR-T cells grown for the same duration did not produce any tumor. A control colon adenocarcinoma cell line C85 was injected as positive control and produced tumors in each mouse.

### REFERENCES

1. Spechler S J, Goyal R K. The columnar-lined esophagus, intestinal metaplasia, and Norman Barrett. Gastroenterology 1996; 110:614-21.
2. Wang K K, Sampliner R E. Updated guidelines 2008 for the diagnosis, surveillance and therapy of Barrett's esophagus. Am J Gastroenterol 2008; 103:788-97.
3. Clemons N J, McColl K E, Fitzgerald R C. Nitric oxide and acid induce doublestrand DNA breaks in Barrett's esophagus carcinogenesis via distinct mechanisms. Gastroenterology 2007; 133:1198-209.
4. Lagergren J, Bergstrom R, Lindgren A, Nyren O. Symptomatic gastroesophageal reflux as a risk factor for esophageal adenocarcinoma. N Engl J Med 1999; 340:825-31.
5. Shaheen N, Ransohoff D F. Gastroesophageal reflux, Barrett esophagus, and esophageal cancer: clinical applications. JAMA 2002; 287:1982-6.
6. Solaymani-Dodaran M, Logan R F, West J, Card T, Coupland C. Risk of extraoesophageal malignancies and colorectal cancer in Barrett's oesophagus and gastro-oesophageal reflux. Scand J Gastroenterol 2004; 39:680-5.
7. Cameron A J, Ott B J, Payne W S. The incidence of adenocarcinoma in columnarlined (Barrett's) esophagus. N Engl J Med 1985; 313:857-9.
8. Wild C P, Hardie L J. Reflux, Barrett's oesophagus and adenocarcinoma: burning questions. Nat Rev Cancer 2003; 3:676-84.
9. Olliver J R, Hardie L J, Dexter S, Chalmers D, Wild C P. DNA damage levels are raised in Barrett's oesophageal mucosa relative to the squamous epithelium of the oesophagus. Biomarkers 2003; 8:509-21.
10. Nehra D, Howell P, Williams C P, Pye J K, Beynon J. Toxic bile acids in gastrooesophageal reflux disease: influence of gastric acidity. Gut 1999; 44:598-602.
11. Morgan C, Alazawi W, Sirieix P, Freeman T, Coleman N, Fitzgerald R. In vitro acid exposure has a differential effect on apoptotic and proliferative pathways in a Barrett's adenocarcinoma cell line. Am J Gastroenterol 2004; 99:218-24.
12. Souza R F, Shewmake K, Terada L S, Spechler S J. Acid exposure activates the mitogen-activated protein kinase pathways in Barrett's esophagus. Gastroenterology 2002; 122:299-307.
13. Jaiswal K, Lopez-Guzman C, Souza R F, Spechler S J, Sarosi G A. Jr. Bile salt exposure increases proliferation through p38 and ERK MAPK pathways in a nonneoplastic Barrett's cell line. Am J Physiol Gastrointest Liver Physiol 2006; 290:G335-42.
14. Jiang Z R, Gong J, Zhang Z N, Qiao Z. Influence of acid and bile acid on ERK activity, PPARgamma expression and cell proliferation in normal human esophageal epithelial cells. World J Gastroenterol 2006; 12:2445-9.
15. Iijima K, Grant J, McElroy K, Fyfe V, Preston T, McColl K E. Novel mechanism of nitrosative stress from dietary nitrate with relevance to gastro-oesophageal junction cancers. Carcinogenesis 2003; 24:1951-60.
16. Debruyne P R, Witek M, Gong L, Birbe R, Chervoneva I; Jin T, Domon-Cell, C. Palazzo, J. P., Freund, J. N., Li, P., Pitari, G. M., Schulz, S., Waldman, S. A. Bile acids induce ectopic expression of intestinal guanylyl cyclase C Through nuclear factor-kappaB and Cdx2 in human esophageal cells. Gastroenterology 2006; 130:1191-206.
17. Kazumori H, Ishihara S, Rumi M A, Kadowaki Y, Kinoshita Y. Bile acids directly augment caudal related homeobox gene Cdx2 expression in oesophageal keratinocytes in Barrett's epithelium. Gut 2006; 55:16-25.
18. Fitzgerald R C, Omary M B, Triadafilopoulos G. Dynamic effects of acid on Barrett's esophagus. An ex vivo proliferation and differentiation model. J Clin Invest 1996; 98:2120-8.
19. Jolly A J, Wild C P, Hardie L J. Acid and bile salts induce DNA damage in human oesophageal cell lines. Mutagenesis 2004; 19:319-24.
20. Dvorak K, Payne C M, Chavarria M, Ramsey L, Dvorakova B, Bernstein H, Holubec, H., Sampliner, R. E., Guy, N., Condon, A., Bernstein, C., Green, S. B., Prasad, A., Garewal, H. S. Bile acids in combination with low pH induce oxidative stress and oxidative DNA damage: relevance to the pathogenesis of Barrett's oesophagus. Gut 2007; 56:763-71.
21. Jenkins G J, D'Souza F R, Suzen S H, Eltahir Z S, James S A, Parry J M, Deoxycholic acid at neutral and acid pH, is genotoxic to oesophageal cells through the induction of ROS: The potential role of anti-oxidants in Barrett's oesophagus. Carcinogenesis 2007; 28:136-42.
22. Das K M, Sakamaki S, Vecchi M, Diamond B. The production and characterization of monoclonal antibodies to a human colonic antigen associated with ulcerative colitis: cellular localization of the antigen by using the monoclonal antibody. J Immunol 1987; 139:77-84.
23. Halstensen T S, Das K M, Brandtzaeg P. Epithelial deposits of immunoglobulin G1 and activated complement colocalise with the M(r) 40 kD putative autoantigen in ulcerative colitis. Gut 1993; 34:650-7.
24. Das K M, Prasad I, Garla S, Amenta P S. Detection of a shared colon epithelial epitope on Barrett epithelium by a novel monoclonal antibody. Ann Intern Med 1994; 120:753-6.
25. DeMeester S R, Wickramasinghe K S, Lord R V, Friedman A, Balaji N S, Chandrasoma P T, Hagen, J. A., Peters, J. H., DeMeester, T. R. Cytokeratin and DAS-1 immunostaining reveal similarities among cardiac mucosa, CIM, and Barrett's esophagus. Am J Gastroenterol 2002; 97:2514-23.
26. Glickman J N, Wang H, Das K M, Goyal R K, Spechler S J, Antonioli D, Odze, R. D. Phenotype of Barrett's esophagus and intestinal metaplasia of the distal esophagus and gastroesophageal junction: an immunohistochemical study of cytokeratins 7 and 20, Das-1 and 45 MI. Am J Surg Pathol 2001; 25:87-94.
27. Piazuelo M B, Hague S, Delgado A, Du J X, Rodriguez F, Correa P. Phenotypic differences between esophageal and gastric intestinal metaplasia. Mod Pathol 2004; 17:62-74.
28. Griffel L H, Amenta P S, Das K M. Use of a novel monoclonal antibody in diagnosis of Barrett's esophagus. Dig Dis Sci 2000; 45:40-8.
29. Rogge-Wolf C, Seldenrijk C A, Das K M, Timmer R, Breumelhof R, Smout A J, Amenta, P. S., Griffel, L. H. Prevalence of mabDAS-1 positivity in biopsy specimens from the esophagogastric junction. Am J Gastroenterol 2002; 97:2979-85.
30. Sefah A, Ang D, Walton K, Das K M. Biological characteristics of cardia type epithelium in patients with Barrett's esophagus. Gastroenterology 2007; 132:A259.
31. Lin J L, Geng X, Bhattacharya S D, Yu J R, Reiter R S, Sastri B, Glazier, K. D. Mirza, Z. K., Wang, K. K.; Amenta, P. S., Das, K. M., Lin, J. J. Isolation and sequencing of a novel tropomyosin isoform preferentially associated with colon cancer. Gastroenterology 2002; 123:152-62.
32. Geng X, Biancone L, Dai H H, Lin J J, Yoshizaki N, Dasgupta A, Pallone, F., Das, K. M. Tropomyosin isoforms in intestinal mucosa: production of autoantibodies to tropomyosin isoforms in ulcerative colitis. Gastroenterology 1998; 114:912-22.
33. Jaiswal K R, Morales C P, Feagins L A, Gandia K G, Zhang X, Zhang H Y, Hormi-Carver, K., Shen, Y., Elder, F., Ramirez, R. D., Sarosi, G. A., Jr., Spechler, S. J., Souza, R. F. Characterization of telomerase-immortalized, non-neoplastic, human Barrett's cell line (BAR-T). Dis Esophagus 2007; 20:256-64.
34. Bajpai M, Liu J, Geng X, Souza R F, Amenta P S, Das K M. Repeated exposure to acid and bile selectively induces colonic phenotype expression in a heterogeneous Barrett's epithelial cell line. Lab Invest 2008; 88:643-51.
35. Rahman L, Voeller D, Rahman M, Lipkowitz S, Allegra C, Barrett J C, Kaye, J., Zajac-Kaye, M. Thymidylate synthase as an oncogene: a novel role for an essential DNA synthesis enzyme. Cancer Cell 2004; 5:341-51.
36. Audrezet M P, Robaszkiewicz M, Mercier B, Nousbaum J B, Hardy E, Bail J P, Volant, A., Lozac'h, P., Gouerou, H., Ferec, C. Molecular analysis of the TP53 gene in Barrett's adenocarcinoma. Hum Mutat 1996; 7:109-13.
37. Keswani R N, Noffsinger A, Waxman I, Bissonnette M. Clinical use of p53 in Barrett's esophagus. Cancer Epidemiol Biomarkers Prey 2006; 15:1243-9.
38. Gonzalez M V, Artimez M L, Rodrigo L, Lopez-Larrea C, Menendez M J, Alvarez V, et al. Mutation analysis of the p53, APC, and p16 genes in the Barrett's oesophagus, dysplasia, and adenocarcinoma. J Clin Pathol 1997; 50:212-7.
39. Wu T T, Watanabe T, Heitmiller R, Zahurak M, Forastiere A A, Hamilton S R. Genetic alterations in Barrett esophagus and adenocarcinomas of the esophagus and esophagogastric junction region. Am J Pathol 1998; 153:287-94.
40. Wong D J, Paulson T G, Prevo L T, Galipeau P C, Longton G, Blount P L, Reid, B. J. p16(INK4a) lesions are common, early abnormalities that undergo clonal expansion in Barrett's metaplastic epithelium. Cancer Res 2001; 61:8284-9.
41. Barrett M T, Sanchez C A, Prevo L J, Wong D J, Galipeau P C, Paulson T G, Rabinovitch, P. S., Reid, B. J., Evolution of neoplastic cell lineages in Barrett oesophagus. Nat Genet 1999; 22:106-9.
42. Katada N, Hinder R A, Smyrk T C, Hirabayashi N, Perdikis G, Lund R J, Woodward, T., Klingler, P. J. Apoptosis is inhibited early in the dysplasiacarcinoma sequence of Barrett esophagus. Arch Surg 1997; 132:728-33.
43. Blount P L, Meltzer S J, Yin J, Huang Y, Krasna M J, Reid B J. Clonal ordering of 17p and 5q allelic losses in Barrett dysplasia and adenocarcinoma. Proc Natl Acad Sci USA 1993; 90:3221-5.
44. Reid B J, Prevo L J, Galipeau P C, Sanchez C A, Longton G, Levine D S, Blount, P. L., Rabinovitch, P. S. Predictors of progression in Barrett's esophagus II: baseline 17p (p53) loss of heterozygosity identifies a patient subset at increased risk for neoplastic progression. Am J Gastroenterol 2001; 96:2839-48.
45. Weston A P, Banerjee S K, Sharma P, Tran T M, Richards R, Cherian R. p53 protein overexpression in low grade dysplasia (LGD) in Barrett's esophagus: inununohistochemical marker predictive of progression. Am J Gastroenterol 2001; 96:1355-62.
46. Zhang H Y, Zhang X, Hormi-Carver K, Feagins L A, Spechler S J, Souza R F. In non-neoplastic Barrett's epithelial cells, acid exerts early antiproliferative effects through activation of the Chk2 pathway. Cancer Res 2007; 67:8580-7.
47. Das-Bhattacharya S, Walton K, Watari J, Amenta P S, Lin J C, Das K M. Expression of a novel human tropomyosin isoform, TC22, in gastric intestinal metaplasia associated with gastric carcinoma. Gastroenterology 2002; 122:A129.
48. Ince T A, Richardson A L, Bell G W, Saitoh M, Godar S, Karnoub A E, Iglehart, J. D., Weinberg, R. A. Transformation of different human breast epithelial cell types leads distinct tumor phenotypes. Cancer Cell 2007; 12:160-70.

1) An in-vitro model for the study of the transformation of non-neoplastic cells to neoplastic cells which comprises treating a benign Barrett's cell line with acid and bile repeatedly over a period of from about 34 weeks to about 84 weeks.

2) The model of claim 1 wherein the Barrett's cell line is BAR-T.

3) The model of claim 1 wherein the cell line is treated for about 65 weeks.

4) The model of claim 1 wherein the cell line is treated with acid and bile daily.

5) The model of claim 1 wherein the cell line is treated with acid and bile for a period of about 5 minutes for each exposure.

6) A process for the study of the transformation of a non-neoplastic cell to a neoplastic cell which comprises
a) growing a Barrett's cell line in medium for a period of from about 34 weeks to about 84 weeks;
b) treating the growing cell line with acid and bile repeatedly
c) harvesting a portion of the cells every 4-6 weeks; and
d) determining the morphological changes, molecular changes and anchorage-independent growth of the cells.

7) The process of claim 6 wherein the cells treated for over about 65 weeks are transplanted into mice and the presence or absence of tumor growth is determined.
